(19) United States
(12) Patent Application Publication
(10) Pub. No.: US 2026/0215745 A1
(43) Pub. Date: Jul. 30, 2026

(54) COLLISION-SENSITIVE HOUSING UNIT, X-RAY DEVICE, AND METHOD OF COLLISION DETECTION

(71) Applicant: Siemens Healthineers AG, Forchheim (DE)

(72) Inventors: Martin Seifert, Bayreuth (DE); Nicole Sommer, Bindlach (DE); Philipp Surm, Fürth (DE)

(21) Appl. No.: 19/463,896

(22) Filed: Jan. 29, 2026

(30) Foreign Application Priority Data
Jan. 29, 2025 (DE) 10 2025 103 161.4

(51) Int. Cl. A61B 6/10; A61B 6/00

(52) U.S. Cl. CPC A61B 6/102; A61B 6/4405; A61B 6/547

(57) ABSTRACT

A collision-sensitive housing has an outer housing layer that is deformable by an external force application. The housing includes a sensor board of an optical force sensor. The outer housing layer and the sensor board are arranged at a predefined spacing from each other, such that a deformation of the outer housing layer causes a change in the spacing. An emitter element of the sensor board is configured to emit a light beam. The light beam is emitted at a predefined angle, such that the light beam is reflected at the outer housing layer and the reflected light beam is deflected onto a receiver element. An evaluation unit is configured to evaluate the reflected light beam with regard to a change in the spacing, and a control unit is configured to interpret the evaluated change in spacing, and to initiate a countermeasure and/or trigger a warning signal.

This application claims the benefit of German Patent Application No. DE 10 2025 103 161.4, filed on Jan. 29, 2025, which is hereby incorporated by reference in its entirety.

### BACKGROUND

The present embodiments relate to a collision-sensitive housing unit, an X-ray device with a collision-sensitive housing unit, and a method of collision detection with a collision-sensitive housing unit.

For movable components of medical devices, such as mounts (e.g., C-arm) or automatically displaceable (e.g., mobile) medical devices, in relation to user and patient safety, facilities for switching the device off in the event of a collision are to be implemented. A large number of different sensors exist for collision detection and avoidance. Up-to-date tactile shut-off apparatuses are used for the different modalities. The implementation of sensor systems of this kind in, for example, a housing part results in many challenges based on the requirements (e.g., surface, single-fault safety, switching paths and forces, etc.) that are to be adhered to. Thus, regimentations of the freedom of design (e.g., radii, shaping faces, cover thickness, industrial design) of the cover part are frequently necessary. The component part costs and the component part weight increases due to laborious implementations. There is an increase in the complexity in relation to component part and manufacturing technology. The integration effort increases. There is a dependence on individual technology carriers. In general, multi-material structures made of thermoplastic polymers, paints, and soft foam components with defined Shore hardness are employed for housings of this kind. The switching function is achieved, by way of example, via conductive polymers, polyurethanes, conductive paints, metallized components, as well as components functionalized in some other way in order to provide electrical conductivity (e.g., wires, meshwork, textiles, etc.), or force sensors. As a rule, the change in resistance/electrical conductivity of the components is used as a measured variable.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, a collision detection apparatus for a partially movable or displaceable medical device that enables simple, inexpensive, and easy-to-implement collision detection is provided. As another example, a method of collision detection with such a collision detection apparatus is provided.

Independent of the grammatical term usage, individuals with male, female, or other gender identities are included within the term.

The present embodiments include a collision-sensitive housing unit for an at least partially movable and/or mobile medical device, having a deformable outer housing layer configured to be deformed by an external application of force, and at least one sensor board of an optical force sensor. The outer housing layer and the sensor board are arranged at a predefined spacing h from one another, such that a deformation of the outer housing layer causes a change in the spacing. The sensor board has a large number of sensor combinations each including an emitter element and at least one associated receiver element. The emitter elements are configured to emit at least one light beam of a specific wavelength. The light beam(s) are emitted at one or more predefined angles a, such that the light beam(s) are reflected at the outer housing layer and the reflected light beam(s) are deflected onto an associated receiver element. The collision-sensitive housing unit includes an evaluation unit with at least one evaluation algorithm, which are configured to evaluate the reflected light beam, received in the receiver element, with regard to a change in spacing of the predefined spacing, and a control unit that is configured to interpret the evaluated change in spacing of the spacing, which overshoots a threshold value, as a collision and as a consequence thereof, to initiate a countermeasure and/or trigger a warning signal.

With a collision-sensitive housing unit of this kind, in the case of medical devices with motorized movable components and/or in the case of self-propelled (e.g., mobile) medical devices, it is possible to detect collisions easily and effectively and take appropriate countermeasures. The collision-sensitive housing unit may be produced with little effort and inexpensively from readily available components and may be easily installed. In addition, no restrictions in relation to the design of the housing unit are necessary. The present embodiments make use of the principle of the optical force sensor, used for operation of controls, in order to detect collisions. For this, the outer housing layer is configured to be deformable. In order to also detect slight collisions, the outer housing layer may be flexibly configured via an appropriate choice of material (e.g., if required, also less flexibly in order to embody the outer housing layer to be less sensitive). A portion of the energy resulting due to the external application of force may also be absorbed by the deformation. With an appropriate choice of material, the outer housing layer may easily be reshaped again. Detection takes place quickly and straightforwardly via the evaluation of the correspondingly reflected and evaluated light beam. A further advantage of the sensor system that is used consists in misdetections due to liquids on the surface of the outer housing layer, which are frequently encountered in a medical environment, not constituting a problem, in contrast, for example, to capacitive surfaces.

According to one embodiment, the collision-sensitive housing unit has an inner housing layer that is configured as a sensor board of the optical force sensor or on which the sensor board is arranged. In this embodiment, the housing unit therefore includes two housing layers, an inner housing layer and an outer housing layer, that form a stable cover for a medical device. Thus, for example, a two-component cover may be provided with an inner housing layer (e.g., hard shell made of compact polymer) and an outer housing layer of, for example, final-coated soft foam component. The force sensor is situated on the hard shell or between hard shell and soft foam component and detects the deformation path of the soft foam component.

According to a further embodiment, the outer housing layer has at its inner side facing the sensor board a reflector or is configured as a reflector. Thus, for example, the inner side of the outer housing layer may be coated with a reflecting material or be configured as a mirror. Such a reflective layer may be installed with little effort and inexpensively.

According to a further embodiment, the collision-sensitive housing unit has a calculating unit that is configured to compare the evaluated change in the spacing with the threshold value. An algorithm may be used for this. Such an algorithm may be easily programmed and employed. A calculation, and therewith collision detection, may take place in real time (e.g., very quickly) so countermeasures may be initiated directly.

According to a further embodiment, one emitter element and one receiver element each are arranged side by side on the sensor board, and the light beam of the emitter element is reflected onto the receiver element situated next to it. In this way, a direct and clear assignment between receiver element and emitter element is possible.

According to a further embodiment, two or four receiver elements are assigned to each emitter element and arranged next to the respective emitter elements, and the emitter element is configured to emit two or four light beams that are reflected onto the corresponding receiver elements. An arrangement may thus be such that an emitter element is surrounded on two opposing sides or all four sides by a receiver element.

According to a further embodiment, the countermeasure is formed by an emergency cutout, a reversal, and/or an evasive movement of the movable and/or mobile medical device. After detection of a collision, further damage or danger to the device may thus be actively counteracted in that, for example, all movements are stopped or further movements in the collision direction are prevented.

According to a further embodiment, the outer housing layer is configured such that an easing of the application of force causes a reshaping into the original undeformed state. In this way, the housing unit is immediately ready after the reshaping, without problems, for further collision detections.

The present embodiments also include an X-ray device (e.g., a motorized C-arm X-ray device) having a collision-sensitive housing unit, as, for example, described above and below. Motorization may move, for example, a C-arm or another movable component. The collision-sensitive housing unit may then be arranged, for example, on the C-arm or the movable component. The X-ray device may be formed, for example, by a stationary, C-arm X-ray device (e.g., suspended from the ceiling or secured to the floor). The X-ray device may also be configured as a CT device with a rotating gantry. The X-ray device may also be configured as a mobile X-ray device (e.g., a mobile, self-propelled motorized C-arm X-ray device with a device cart).

The present embodiments also include a method of collision detection using a collision-sensitive housing unit as described above, with the following acts: emission of at least one light beam of at least one predefined wavelength by at least one emitter element of the large number of emitter elements of the optical force sensor board; receiving of an external application of force onto the deformable outer housing layer and consequently deforming of the outer housing layer; receiving of at least one light beam reflected at a reflector of the deformed housing layer by at least one receiver element; evaluating the reflected light beam with regard to a change in the spacing; comparing the change in the spacing with a threshold value; and when the threshold value is overshot, initiating a countermeasure and/or triggering a warning signal. According to one embodiment, an emergency cutout, a reversal, and/or an evasive movement of the movable and/or mobile medical device is carried out as a countermeasure.

### BRIEF DESCRIPTION OF THE DRAWINGS

The invention as well as further embodiments are explained in more detail below in the drawings based on schematically represented example embodiments, without the invention being limited to these example embodiments thereby. In the drawings:

FIG. 1 shows a perspective view of a detail of a collision-sensitive housing unit with an emitter element and a receiver element;

FIG. 2 shows a sequence of acts of an embodiment of a method of collision detection;

FIG. 3 shows a sectional view of a detail of a collision-sensitive housing unit with an emitter element and a receiver element in the basic state;

FIG. 4 shows a sectional view of a detail of a collision-sensitive housing unit with an emitter element and a receiver element in the deformed state;

FIG. 5 shows a sectional view of a detail of a further collision-sensitive housing unit with an emitter element and a receiver element with an inner housing layer;

FIG. 6 shows a sectional view of a detail of a further collision-sensitive housing unit with an emitter element and two receiver elements;

FIG. 7 shows the plotting of a typical dependency between luminous intensity and spacing; and

FIG. 8 shows a C-arm X-ray device with a collision-sensitive housing unit.

### DETAILED DESCRIPTION

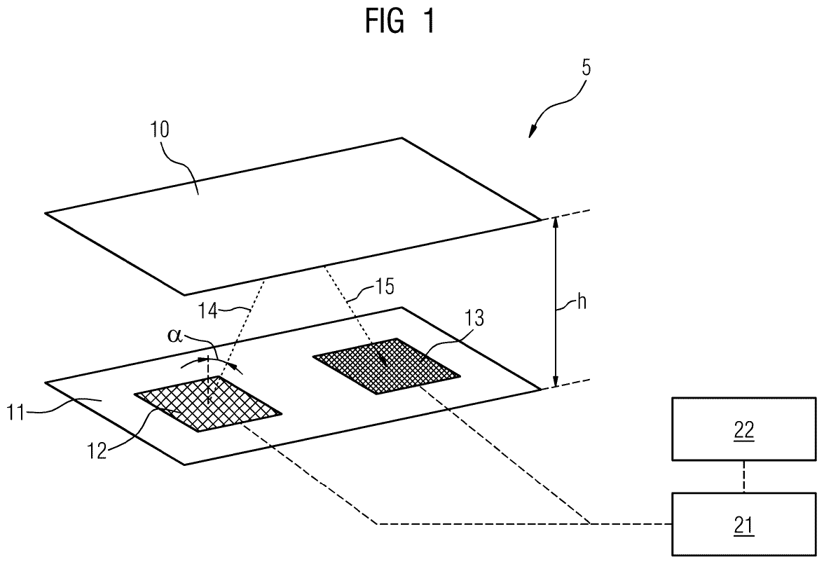

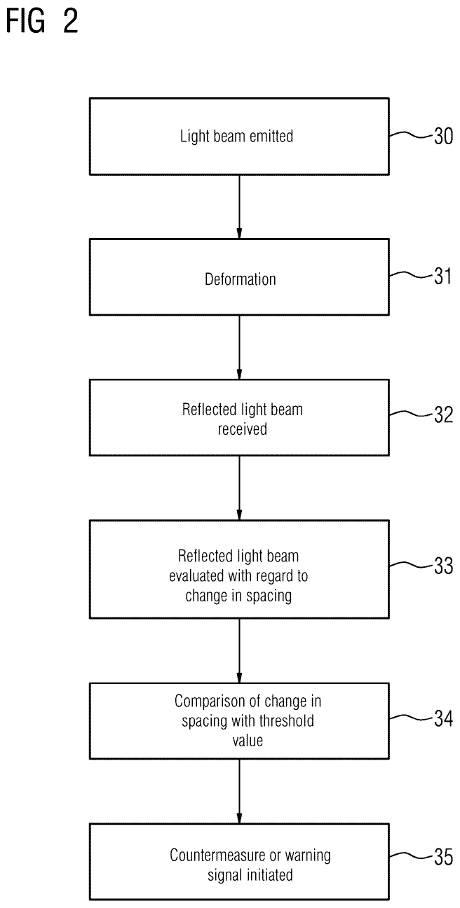

FIG. 1 shows a perspective section of a detail of a collision-sensitive housing unit 5, having an outer housing layer 10 and a sensor board 11 with an emitter element 12 and a receiver element 13. The outer housing layer 10 is formed from a flexible material that may be deformed on application of force (e.g., concavely in a direction of the sensor board 11). The deformation may be reversible (e.g., as soon as the application of force ceases). In general, a collision-sensitive housing unit 5 has a large number of emitter elements 12 and receiver elements 13. The mode of operation may be shown using a single pair of sensors. The emitter element 12 is configured to emit at least one light beam 14, with the light beam having a specified wavelength. Typical wavelengths may lie, for example, in the visible range (e.g., between 400 to 700 nm). The angle a, at which the light beam 14 is emitted, is selected such that the light beam 15 reflected at the lower side of the outer housing layer 10 (e.g., at a reflector 17 arranged there or a coating on the inner side of the outer housing layer facing the emitter element) strikes the receiver element 13. The sensor board 11 (or alternatively the emitter element 12 and the receiver element 13) has a predefined (e.g., set up during manufacture or production of the housing) spacing h from the outer housing layer 10; this may be, for example, 1 or 2 mm or also be selected to be larger or smaller in relation to the sensor function. The receiver element 13 detects the reflected light beam 15. An evaluation unit 21 for ascertaining the change in spacing D is connected to the sensor board 11. This is connected to an actuation unit 22. Overall, the described unit represents only a detail of a collision-sensitive housing unit since the entire apparatus may have a large number of emitter elements 12 and receiver elements 13. For this, for example, the sensor board 11 may have a larger surface, or a large number of sensor boards 11 may be arranged side by side.

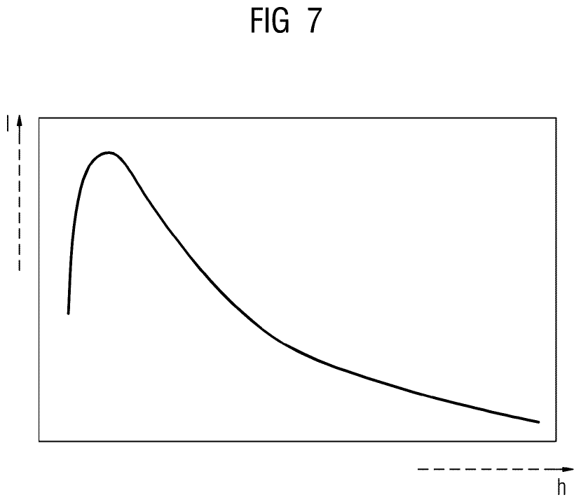

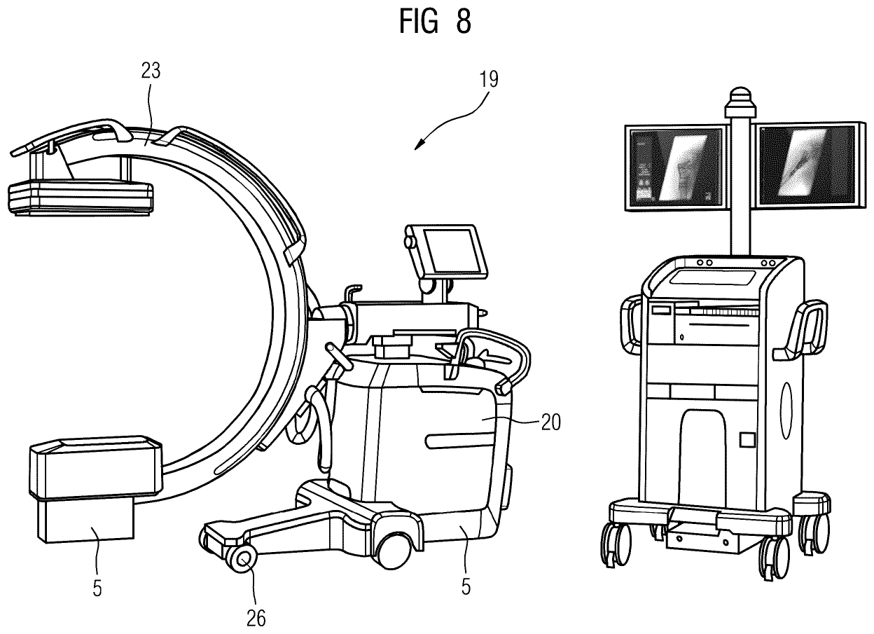

The collision-sensitive housing unit 5 is part of a, for example, mobile medical device, such as a mobile motorized C-arm X-ray device 19 (see, e.g., FIG. 7) that, using wheels 26, may be displaced on the ground manually or automatically in a motorized manner. In one embodiment, the collision-sensitive housing unit may, for example, include the entire housing or a detail of the housing. Thus, for example, a detail that is particularly affected by collisions, such as side parts and/or other sections of the device cart 20 or parts of the C-arm 23, such as the covering of the X-ray tube 24 or the X-ray detector 25, may be configured as the collision-sensitive housing unit. The collision-sensitive housing unit is configured to effectively detect collisions in good time, so a fast reaction is possible, and thus, damage to devices and/or individuals may be prevented.

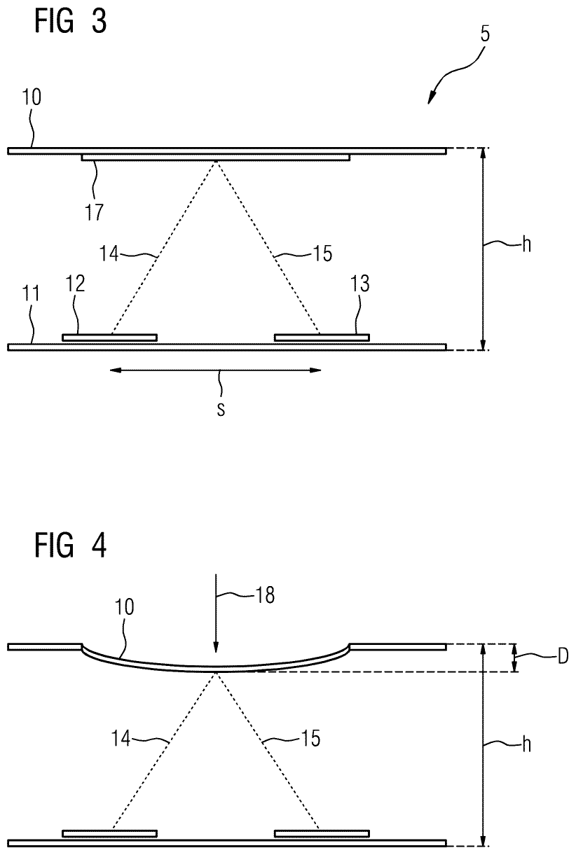

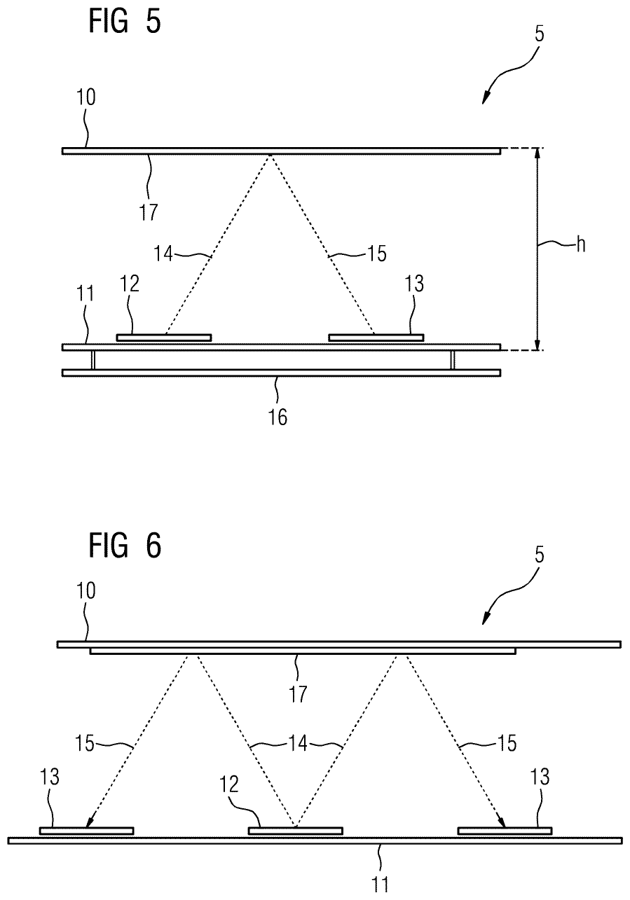

FIGS. 3, 5, and 6 show further examples of details of collision-sensitive housing units 5. In FIG. 3, similar to as in FIG. 1, an emitter element 12 and a receiver element 13 are assigned to one another as a pair of sensors, have an emitter-receiver spacing s from one another, and the respective emitter element 12 emits a light beam such that the reflected light beam falls on the assigned receiver element exactly. In FIG. 6, two receiver elements 13 are assigned to one emitter element 12, and the emitter element 12 emits two light beams in different directions (e.g., at the angles a and −a with respect to the normal to the sensor board 11), which are reflected onto the two receiver elements 13 adjacent to the emitter element 12. The respective emitter-receiver spacings s may be identical. The two light beams 14 may have the same or different predefined wavelengths. Reflectors 17 are arranged on the respective inner side of the outer housing layer 10. The reflector(s) 17 are secured to the inner side (e.g., glued, screwed, etc.), or the inner side of the outer housing layer 10 may also be coated so as to be reflective (e.g., with optically reflective paints, metallic or non-metallic reflective compounds, or reflective films). The reflector(s) 17 may extend over the entire surface of the surface of the outer housing layer facing the sensor board. In FIG. 5, the sensor board 11 is connected to an inner housing layer 16 (e.g., may be connected to the inner housing layer 16 using connecting elements, or may be secured to the inner housing layer 16 directly, such as glued, screwed, etc.).

FIG. 4 shows the mode of operation of the collision detection. If the flexible outer housing layer 10 is deformed or dented in the direction of the sensor board 11 due to an external application of force 18, such as an impact or a collision with another object or an individual, then the predefined spacing h between the sensor board 11 and the outer housing layer 10 is shortened by a change in spacing D. The light beam 13 received by the receiver element 15 also changes thereby (e.g., with regard to the luminous intensity). This changed luminous intensity may be evaluated by the evaluation unit, with it being possible for the evaluation to also ascertain a value for the change in spacing D. An algorithm employed or developed for this may compare, for example, the measured value with the original values before the change, and output a value for the change in spacing D or a value proportional to the change in spacing D. FIG. 7 shows an illustration of the correlation between the spacing h and the luminous intensity I. With a known wavelength and known emitter-receiver spacing s, the change in spacing D may be determined from the original luminous intensity and the changed luminous intensity (e.g., using a previously determined dependency or a formula). An example deformation, which with a predefined spacing h may still be reliably measured and evaluated with such a sensor, is, for example, D ~1 μm. Therefore, very slight changes in spacing D that were generated by very slight collisions may already be measured. A corresponding coordination between the flexibility of the outer housing layer and the structure and the equipping of the sensor board may therefore provide particularly sensitive collision detection.

FIG. 3 shows acts of a method of collision detection using the collision-sensitive housing unit described above. In a first act 30, at least one light beam of at least one predefined wavelength is emitted by at least one emitter element of the large number of emitter elements of the optical sensor board. In one embodiment, a lot of or all of the emitter elements are in operation. The emitted wavelength may be the same for all emitter elements (e.g., single wavelength), or optionally, two or more wavelengths (e.g., dual or multiple wavelength) may also be used. The emitter elements may be permanently in operation or be actively switched under specific conditions (e.g., during operation of the medical device or if the medical device moves/is displaced).

If a collision occurs, then in a second act 31, an external application of force is received by the deformable outer housing layer, and this causes a deformation of the outer housing layer. The deformation may be dependent on the flexibility or deformability of the outer housing layer. The deformation may look, for example, like that in FIG. 4. As a result of deformation, the reflector moves closer to the sensor board and the path of the light beam shortens, just like the path of the reflected light beam. The luminous intensity, which the affected receiver element measures in a third act 32, consequently changes. In a fourth act 33, the changed luminous intensity is subsequently evaluated, and the change in spacing D is determined from the changed luminous intensity. In order, for example, to avoid an over-sensitivity or errors due to tiny variations in the luminous intensity, the change in spacing D may subsequently be compared in a fifth act 34 with a threshold value. If the change in spacing D overshoots the threshold value, then in a sixth act 35, a countermeasure is initiated and/or a warning signal is triggered. For example, a control unit for actuation may be used for this, to which the information about the collision is transferred. The control unit then actuates, for example, an output unit (e.g., an optical output unit, such as a monitor or a touchpad or an acoustic output unit, such as a microphone). The corresponding output unit outputs, for example, an image display or text display or a warning tone or a flashing light. Alternatively or in addition, the control unit actuates an emergency cutout, a reversal, and/or an evasive movement of the movable and/or mobile medical device. Thus, for example, if a collision is detected when the device cart of the mobile C-arm X-ray device moves, the device cart may be actuated to stop, reverse, or bypass the obstacle.

Within the framework of the described collision-sensitive housing unit, a concept that was actually developed for controls is transferred to the principle of action of a tactile cover for collision detection. A deformation of specific elements of the cover part in the case of collision (e.g., application of force) results in a switching function that is used, for example, for collision detection and countermeasure.

A housing that has such a collision-sensitive housing unit or is constructed from such a collision-sensitive housing, may be configured, for example, as follows.

For example, a single-shell, monolithic housing with an outer housing layer made of rigid foam or compact polymer (e.g., thermoforming or injection molding) may be used with a low level of complexity (e.g., in relation to free-form surfaces, radii, etc.). The outer housing layer has a defined Shore hardness and material thickness of the polymer, which provides a correspondingly defined deformability. One or more sensor boards with the corresponding emitter elements and receiver elements is then arranged below the outer housing layer at a predefined spacing h. A collision may be detected via deformation on application of force.

The housing may also be formed by a two-component cover made of an internal hard shell made of compact polymer (e.g., thermoforming or injection molding) as the inner housing layer and final-coated soft foam component with defined Shore hardness as the outer housing layer. The sensor board(s) are situated between hard shell and soft foam component and detect the deformation path of the soft foam component. More complex component parts may be implemented thereby, and collision functions may be implemented above all in edge regions or convex/concave surfaces.

Many advantages result from the application of this sensor principle owing to the possibility of fastening a plurality of sensors directly to a single-shell cover part and thus making it possible to measure the deformations. The complexity and costs for a cover for collision detection may thus be reduced by a collision-sensitive housing unit. Liquids on the surface (e.g., compared to capacitive surfaces) of such a housing unit do not affect collision detection (e.g., in contrast to capacitive surfaces). To provide the functionality, as far as possible, there should be no coatings (e.g., conductive paints, spacers) present in the cover. This also increases the recyclability. In addition, much thinner material thicknesses and thus a lower cover weight may be used. Due to the possibility of influencing the materials on application with regard to mechanical properties, there are significantly fewer restrictions in relation to the cover design and the integration of the cover.

The present embodiments may be briefly summarized as follows: For particularly simple and effective detection of collisions even in the case of movable and/or mobile medical devices, a collision-sensitive housing unit is provided. The collision-sensitive housing unit has a deformable outer housing layer that is configured so the deformable outer housing layer may be deformed by an external application of force, and at least one sensor board of an optical force sensor. The outer housing layer and the sensor board are arranged at a predefined spacing from each other, such that a deformation of the outer housing layer causes a change in the spacing. The optical force sensor board has a large number of sensor combinations (e.g., sensor pairs), each including an emitter element and at least one associated receiver element. The emitter elements are configured to emit at least one light beam of a specific wavelength. The light beam(s) are emitted at one (or more) predefined angle(s) a, such that the light beam(s) are reflected at the (e.g., inner side of the) outer housing layer, and the reflected light beam(s) are deflected onto an associated receiver element. The collision-sensitive housing unit includes an evaluation unit with at least one evaluation algorithm. The evaluation unit is configured to evaluate the reflected light beam, received in the receiver element, with regard to the change in spacing. The collision-sensitive housing unit includes a control unit configured to interpret an evaluated change in spacing, which overshoots a threshold value, as a collision, and as a consequence thereof, to initiate a countermeasure and/or trigger a warning signal.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A collision-sensitive housing unit for an at least partially movable, mobile, or movable and mobile medical device, the collision-sensitive housing unit comprising:
an outer housing layer that is deformable by an external application of force;
a sensor board of an optical force sensor, wherein the outer housing layer and the sensor board are arranged at a predefined spacing from each other, such that a deformation of the outer housing layer causes a change in the spacing, wherein the sensor board has a number of sensor combinations, each comprising an emitter element and at least one associated receiver element, wherein the emitter elements are configured to emit at least one light beam of a specific wavelength, and wherein the at least one light beam is emitted at one or more predefined angles, such that the at least one light beam is reflected at the outer housing layer and the at least one reflected light beam is deflected onto an associated receiver element;
an evaluation unit comprising at least one evaluation algorithm, the evaluation unit being configured to evaluate the reflected light beam, received in the receiver element, with regard to a change in spacing of the predefined spacing; and
a controller configured to:
interpret the evaluated change in the spacing, which overshoots a threshold value, as a collision; and
as a consequence of the interpretation, initiate a countermeasure, trigger a warning signal, or initiate the countermeasure and trigger the warning signal.

2. The collision-sensitive housing unit of claim 1, further comprising an inner housing layer configured as the sensor board or on which the sensor board is arranged.

3. The collision-sensitive housing unit of claim 1, wherein the outer housing layer has, at an inner side facing the sensor board, a reflector, or is configured as the reflector.

4. The collision-sensitive housing unit of claim 1, further comprising a calculating unit that is configured to compare the evaluated change in spacing of the spacing with the threshold value.

5. The collision-sensitive housing unit of claim 1, wherein one emitter element and one receiver element each are arranged side by side on the sensor board, and the light beam of the emitter element is reflected onto the receiver element situated next to the emitter element.

6. The collision-sensitive housing unit of claim 1, wherein two or four receiver elements are assigned to each emitter element and are arranged next to the respective emitter element, and
wherein the respective emitter element is configured to emit two or four light beams that are reflected onto the corresponding receiver elements.

7. The collision-sensitive housing unit of claim 1, wherein the controller is configured to initiate the countermeasure, and
wherein the countermeasure is formed by an emergency cutout, a reversal, an evasive movement of the movable, mobile, or movable and mobile medical device, or any combination thereof.

8. The collision-sensitive housing unit of claim 1, wherein the outer housing layer is configured such that an easing of the application of force causes a reshaping into an original undeformed state.

9. An X-ray device comprising:
a collision-sensitive housing unit comprising:
an outer housing layer that is deformable by an external application of force;
a sensor board of an optical force sensor, wherein the outer housing layer and the sensor board are arranged at a predefined spacing from each other, such that a deformation of the outer housing layer causes a change in the spacing, wherein the sensor board has a number of sensor combinations, each comprising an emitter element and at least one associated receiver element, wherein the emitter elements are configured to emit at least one light beam of a specific wavelength, and wherein the at least one light beam is emitted at one or more predefined angles, such that the at least one light beam is reflected at the outer housing layer and the at least one reflected light beam is deflected onto an associated receiver element;
an evaluation unit comprising at least one evaluation algorithm, the evaluation unit being configured to evaluate the reflected light beam, received in the receiver element, with regard to a change in spacing of the predefined spacing; and
a controller configured to:
interpret the evaluated change in spacing of the spacing, which overshoots a threshold value, as a collision; and
as a consequence of the interpretation, initiate a countermeasure, trigger a warning signal, or initiate the countermeasure and trigger the warning signal.

10. The X-ray device of claim 9, wherein the X-ray device is an at least partially movable, mobile, or movable and mobile C-arm X-ray device.

11. A method of collision detection using a collision-sensitive housing unit for an at least partially movable, mobile, or movable and mobile medical device, the method comprising:
emitting at least one light beam of at least one predefined wavelength by at least one emitter element of a number of emitter elements of a sensor board of an optical force sensor, the collision-sensitive housing unit comprising the sensor board;
receiving an external application of force onto a deformable outer housing layer of the collision-sensitive housing unit, and consequently, deforming the deformable outer housing layer;
receiving at least one light beam reflected at a reflector of the deformed housing layer by at least one receiver element;
evaluating the reflected light beam with regard to a change in spacing;
comparing the change in the spacing with a threshold value; and
when the threshold value is overshot, initiating a countermeasure, triggering a warning signal, or a combination thereof.

12. The method of claim 11, wherein the collision-sensitive housing unit comprises:
the outer housing layer, which is deformable by the external application of force;
the sensor board of the optical force sensor, wherein the outer housing layer and the sensor board are arranged at a predefined spacing from each other, such that a deformation of the outer housing layer causes the change in the spacing, wherein the sensor board has a number of sensor combinations, each comprising an emitter element and at least one associated receiver element, wherein the emitter elements are configured to emit at least one light beam of a specific wavelength, and wherein the at least one light beam is emitted at one or more predefined angles, such that the at least one light beam is reflected at the outer housing layer and the at least one reflected light beam is deflected onto an associated receiver element;
an evaluation unit comprising at least one evaluation algorithm, the evaluation unit being configured to evaluate the reflected light beam, received in the receiver element, with regard to the change in spacing of the predefined spacing; and
a controller configured to:
interpret the evaluated change in the spacing, which overshoots the threshold value, as a collision; and
as a consequence of the interpretation, initiate the countermeasure, trigger the warning signal, or initiate the countermeasure and trigger the warning signal.

13. The method of claim 11, wherein when the threshold value is overshot, initiating the countermeasure, triggering the warning signal, or the combination thereof comprises carrying out, as the countermeasure, an emergency cutout, a reversal, an evasive movement, or the reversal and the evasive movement of the movable, mobile, or movable and mobile medical device, or any combination thereof.
